(19) United States
(12) Patent Application Publication
(10) Pub. No.: US 2011/0301314 A1
(43) Pub. Date: Dec. 8, 2011

(54) CONTINUOUS LIQUID-PHASE PROCESS FOR THE SYNTHESIS OF DIAMINOPYRIDINES FROM GLUTARONITRILES

(75) Inventors: Keith W. Hutchenson, Lincoln University, PA (US); Aaron Minter, Wilmington, DE (US)

(73) Assignee: E.I. DU PONT DE NEMOURS AND COMPANY, Wilmington, DE (US)

(21) Appl. No.: 13/131,332

(22) PCT Filed: Dec. 17, 2009
(86) PCT No.: PCT/US09/68435

§ 371 Date: Aug. 16, 2011

Related U.S. Application Data
(60) Provisional application No. 61/138,792, filed on Dec. 18, 2008.

(51) Int. Cl. C08F 22/40; C07D 213/73

(52) U.S. Cl. 526/262; 546/250

(57) ABSTRACT

A liquid-phase, continuous process is provided for the manufacture of 2,6-diaminopyridine and related compounds from glutaronitriles, which are used industrially as compounds and as components in the synthesis of a variety of useful materials. The synthesis proceeds by means of a dehydrogenative aromatization process.

This application claims priority under 35 U.S.C. §119(e) from, and claims the benefit of, U.S. Provisional Application No. 61/138,792, filed Dec. 18, 2008, which is by this reference incorporated in its entirety as a part hereof for all purposes.

### TECHNICAL FIELD

This invention relates to the manufacture of diaminopyridines and related compounds, and to the industrial use thereof for the synthesis of other useful materials.

### BACKGROUND

The compound 2,6-diaminopyridine (“DAP”), which is represented by the structural formula shown below:

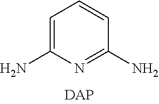

is a useful starting material for preparing monomers for rigid rod polymers such as those described in WO 94/25506, as well as for the preparation of dyes, metal ligands, medicines and pesticides.

It is known to prepare DAP by means of the Chichibabin amination reaction, in which pyridine is reacted with sodium amide in an organic solvent. This is a complicated reaction, however, and handling the sodium amide and isolating the desired product from this complex mixture are difficult operations to perform on a commercial scale.

The synthesis of 2,6-diaminopyridine and related compounds from glutaronitriles or glutarimidines may be described as proceeding via a dehydrogenative aromatization reaction. A batch process for the preparation of 2,6-diaminopyridine and related compounds from glutaronitriles and related compounds by contacting an acyclic dinitrile compound with a chemical oxidant and/or a dehydrogenation catalyst in liquid ammonia neat or in a mixture of ammonia and a polar, aprotic solvent, and heating the reaction mixture in a closed vessel, is described in U.S. application Ser. No. 12/519,592, filed Jun. 17, 2009 (previously published as WO 2008/82509 and published as U.S. Patent Publication No. 20/______), which is by this reference incorporated in its entirety as a part hereof for all purpose.

A batch process for the preparation of 2,6-diaminopyridine and related compounds from glutarimidines and related compounds by contacting a glutarimidine with a chemical oxidant and/or a dehydrogenation catalyst in liquid ammonia neat or in a mixture of ammonia and a polar, aprotic solvent, and heating the reaction mixture in a closed vessel, is described in U.S. application Ser. No. 12/516,005, filed May 22, 2009 (previously published as WO 2008/82500 and published as U.S. Patent Publication No. 20/______), which is by this reference incorporated in its entirety as a part hereof for all purpose.

A continuous gas-phase process for the preparation of 2,6-diaminopyridine and related compounds from glutaronitriles and related compounds by contacting an acyclic dinitrile compound in the form of a gas with a dehydrogenation catalyst and heating in the presence of ammonia gas or a mixture of ammonia gas and a carrier gas, is described in U.S. patent application Ser. No. 12/169,152 (filed 8 Jul. 2008 and published as U.S. Patent Publication No. 20/______), which is by this reference incorporated in its entirety as a part hereof for all purpose.

Despite these existing processes to make aminopyridines, a need remains for a process for the continuous liquid-phase preparation of aminopyridines, and in particular DAP and related compounds. This would allow the reaction to be run at lower temperatures and/or higher pressure which may increase the productivity of the process.

### SUMMARY

The inventions disclosed herein include processes for the preparation of diaminopyridines and related compounds, processes for the preparation of products into which diaminopyridines and related compounds can be converted, and the products obtained and obtainable by such processes.

Features of certain of the processes of this invention are described herein in the context of one or more specific embodiments that combine various such features together. The scope of the invention is not, however, limited by the description of only certain features within any specific embodiment, and the invention also includes (1) a subcombination of fewer than all of the features of any described embodiment, which subcombination may be characterized by the absence of the features omitted to form the subcombination; (2) each of the features, individually, included within the combination of any described embodiment; and (3) other combinations of features formed by grouping only selected features taken from two or more described embodiments, optionally together with other features as disclosed elsewhere herein. Some of the specific embodiments of the processes hereof are as follows:

In one embodiment hereof, this invention provides a continuous process for the synthesis of a compound as represented by the structure of the following

Formula (I)

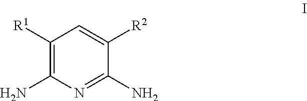

by (a) providing a compound as represented by the structure of the following Formula (II)

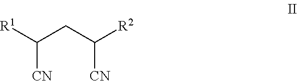

in the form of a liquid; (b) providing an ammonia component selected from the group consisting of: neat liquid ammonia, a mixture of liquid ammonia and a solvent, and ammonia gas; (c) heating a heterogeneous dehydrogenation catalyst; and (d) contacting the Formula II compound and the ammonia component in the presence of the catalyst to produce a Formula (I) product; wherein R¹ and R² are each independently selected from H and a hydrocarbyl group.

In another embodiment hereof, this invention provides a process for preparing a Formula (I) compound, as described above, that further includes a step of subjecting the Formula (I) compound to a reaction (including a multi-step reaction) to prepare therefrom a compound (such as that useful as a monomer), oligomer or polymer.

An advantageous feature of the processes hereof is that they are conducted in the liquid phase in a continuous manner, thereby resulting in a significant decrease in overall reaction time in a liquid phase process and enabling component recycle without isolation. For example, the process may be carried out at low temperatures, e.g. a temperature of about 160° C., at short reaction times. Such features combine to produce an economically favorable process.

### BRIEF DESCRIPTION OF THE DRAWINGS

Various features and/or embodiments of this invention are illustrated in a drawing as described below. These features and/or embodiments are representative only, and the selection of these features and/or embodiments for inclusion in the drawing should not be interpreted as an indication that subject matter not included in the drawing is not suitable for practicing the invention, or that subject matter not included in the drawing is excluded from the scope of the appended claims and equivalents thereof.

FIG. 1 is a schematic representation of a reactor as may be used in a process hereof.

### DETAILED DESCRIPTION

In a process as described herein, there is provided a continuous process for the liquid-phase preparation of 2,6-diaminopyridine and related compounds from glutaronitrile and related compounds.

In one embodiment of the processes hereof, a diaminopyridine compound [as represented by the structure of Formula (I) as shown below] may be synthesized from an acyclic dinitrile compound [as represented by the structure of Formula (II) as shown below] by providing the acyclic dinitrile compound in the form of a liquid; providing an ammonia component selected from the group consisting of: neat liquid ammonia, a mixture of liquid ammonia and a solvent and ammonia gas; heating a dehydrogenation catalyst; and contacting the dinitrile compound and the ammonia component in the presence of the heated catalyst to produce the desired diaminopyridine [Formula (I)] product.

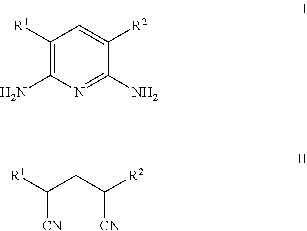

In Formulae (I) and (II), R¹ and R² are each independently selected from H, and a hydrocarbyl group. Examples of hydrocarbyl groups suitable for use in R¹ or R² include without limitation

a C₁˜C₁₂, C₁˜C₈, C₁˜C₆, or C₁˜C₄, straight-chain or branched, saturated or unsaturated, substituted or unsubstituted, aliphatic hydrocarbyl group; and

a C₃˜C₁₂, C₃˜C₈, or C₃˜C₆, cyclic, saturated or unsaturated, substituted or unsubstituted, aliphatic hydrocarbyl group.

An unsubstituted hydrocarbyl group as described above contains no atoms other than carbon and hydrogen. In a substituted hydrocarbyl group,

one or more heteroatoms selected from O, N, S and P may optionally be substituted for any one or more of the in-chain (i.e. non-terminal) or in-ring carbon atoms, provided that each heteroatom is separated from the next closest heteroatom by at least one and preferably two carbon atoms, and that no carbon atom is bonded to more than one heteroatom; and/or

one or more halogen atoms may optionally be bonded to a terminal carbon atom.

In addition, however, a substituted C₃˜C₁₂ cyclic hydrocarbyl group may contain one or more C₁˜C₈, or C₁˜C₄, straight-chain or branched, saturated or unsaturated, aliphatic hydrocarbyl groups bonded to a carbon atom in the ring structure, such group itself optionally being substituted with one or more heteroatoms selected from O, N, S and P, and/or containing one or more halogen atoms, subject to the conditions set forth above.

A C₁˜C₁₂ straight-chain or branched, saturated or unsaturated, substituted or unsubstituted, aliphatic hydrocarbyl group suitable for use herein may include, for example, a methyl, ethyl, n-propyl, i-propyl, n-butyl, sec-butyl, tert-butyl, n-pentyl, n-hexyl, n-octyl, trimethylpentyl, allyl and/or propargyl group. An unsaturated aliphatic group may include one or more double bonds, such as in a dienyl or terpenyl structure, or a triple bond such as in an acetylenyl structure. A C₃˜C₁₂ cyclic, saturated or unsaturated, substituted or unsubstituted, aliphatic hydrocarbyl group suitable for use herein may include, for example, an alicyclic functional group containing in its structure, as a skeleton, cyclohexane, cyclooctane, norbornane, norbornene, perhydro-anthracene, adamantane, or tricyclo-[5.2.1.0^{2.6}]- decane groups. Preferably, one or both of R¹ and R² are H.

When R¹ and R² are both H, the acyclic dinitrile is glutaronitrile (“GN”) and the Formula (I) compound is 2,6-diaminopyridine (“DAP”), as represented by the structures of the formulae shown in the following reaction scheme:

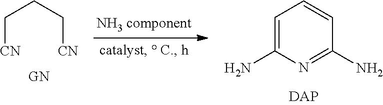

Various compounds of Formula (II), for use as a starting material herein, may be synthesized by processes known in the art, or are available commercially from suppliers such as Alfa Aesar (Ward Hill, Mass.), City Chemical (West Haven, Conn.), Fisher Scientific (Fairlawn, N.J.), Sigma-Aldrich (St. Louis, Mo.) or Stanford Materials (Aliso Viejo, Calif.).

In the processes hereof, a Formula (II) acyclic dinitrile compound in the form of a liquid is contacted with an ammonia component selected from the group consisting of: neat liquid ammonia, a mixture of liquid ammonia and a solvent and ammonia gas.

The reaction is conducted in the liquid phase, i.e. the reaction temperature and pressure are selected to ensure a liquid state for the Formula (II) compound-rich phase by operating at a temperature less than the boiling point of the particular Formula (II) compound utilized in the reaction under the chosen reaction conditions. For example, the boiling point of the Formula (II) compound when it is glutaronitrile (i.e. R¹ and R² are both H) is 286° C. at atmospheric pressure (1 atm, 0.101 MPa), 319° C. at 2 atm (0.203 MPa), 370° C. at 5 atm (0.507 MPa), 392° C. at 7 atm (0.709 MPa), and 417° C. at 10 atm (1.01 MPa) [as described in Design Institute for Physical Properties (DIPPR®) 2004]. Therefore at, for example, 2 atm (0.203 MPa) pressure, the selected reaction temperature would be below 319° C. to maintain the glutaronitrile-rich phase as a liquid.

In a process hereof, a Formula (II) compound in the form of a liquid is contacted with an ammonia component in the presence of a heterogeneous dehydrogenation catalyst. A catalyst suitable for use in a process hereof is a substance that increases the rate of approach to equilibrium of the reaction without itself being substantially consumed in the reaction. A dehydrogenation catalyst suitable for use herein typically contains at least one metal, or metal salt, wherein the metal for use in the catalyst is selected, for example, from elements of Groups IVA, VA, VIA, VIIA, VIII, IB and/or IIB of the Periodic Table [as such groups are described, for example, in the periodic table in a reference such as Advanced Inorganic Chemistry by Cotton and Wilkinson, Interscience, New York, 2nd Ed. (1966)]. A particular metal, for use by itself or in a metal salt, may be selected from Group VIII elements such as iron, cobalt and nickel; and/or from the platinum group of metals including ruthenium, rhodium, palladium, osmium, iridium and platinum. The platinum group of metals and their salts are preferred, more preferably platinum and palladium and their salts. Sponge metal catalysts may also be used, including without limitation Raney® iron, Raney® nickel, Raney® cobalt (Raney is a registered trademark of W.R. Grace and Company, Columbia Md. USA) and equivalent sponge metal catalysts.

In a heterogeneous catalyst, a metal or metal salt of the desired elements may be deposited on any support with a sufficiently high surface area. A heterogeneous catalyst may thus be distinguished from a homogeneous catalyst, which is not supported, in the sense that a homogeneous catalyst and the reactants reside in the same phase, which is uniform, and the catalyst is molecularly dispersed with the reactants in that phase.

The support for a heterogeneous catalyst as used herein may be amorphous or may possess a crystalline structure, or may contain both amorphous and crystalline portions. The support may be a solid metal oxide or solid non-metal oxide, each with surface —OH groups. Examples of such metal oxides are those from tri- and tetravalent metals, which may be a transition or non-transition metal or any rare earth such as alumina, titania, cobaltic oxide, zirconia, ceria, molybdenum oxide and tungsten oxide. An example of a typical non-metal oxide is silica. The support may also be a zeolite or zeotype material having a structure made up of tetrahedra joined together through oxygen atoms to produce an extended network with channels of molecular dimensions. The zeolite/zeotype materials typically have SiOH and/or AlOH groups on the external or internal surfaces. The support may also be activated carbon, coke or charcoal. Preferably, the support is at least one of alumina, silica, silicalite, ceria, titania, or carbon, more preferably alumina, silica or carbon.

In one embodiment of the processes hereof, the reaction is conducted by injecting a Formula (II) dinitrile compound in liquid form, and liquid ammonia neat or in a mixture of liquid ammonia and a solvent, as reactants in liquid form into a reactor that is loaded with the desired catalyst. The Formula (II) dinitrile compound may be supplied neat or in a solution. Suitable solvents for the Formula (II) dinitrile compound include without limitation ethanol, 1,4-dioxane, tetrahydrofuran and acetone. Mixed solvents can be used. Ethanol is preferred as a solvent for the Formula (II) dinitrile compound. Where the ammonia component is a mixture of liquid ammonia and a solvent, suitable solvents for that purpose include without limitation 1,4-dioxane, tetrahydrofuran, acetone, acetonitrile, dimethylformamide and pyridine. Mixed solvents can also be used, such as 1,4-dioxane plus pyridine.

In a further embodiment of the processes hereof, the ammonia component is in the form of a gas. Ammonia, as anhydrous ammonia, has a boiling point of about −33° C., and is therefore available as a gas at ambient temperatures, and may be used as such for injection into the reactor.

The reactions hereof are conducted in the liquid phase, i.e. the reaction temperature and pressure are selected to ensure a liquid state for the Formula (II) compound-rich phase by operating at a temperature less than the boiling point of the particular Formula (II) compound utilized in the reaction under the chosen reaction conditions. The reaction can be conducted in the liquid phase at a temperature that may suitably be about 125° C. or more, or about 150° C. or more, and yet about 300° C. or less, or about 200° C. or less, or about 175° C. or less; or that may be in the range of from about 125° C. to about 300° C., in the range of from about 125° C. to about 200° C., or in the range of from about 150° C. to about 175° C. The reaction temperature referred to here is the temperature that has been provided for the catalyst in the catalyst zone of the reactor. A temperature in these ranges is provided by heating the various portions of the reactor from a source external thereto, in particular a heating element designed to surround and heat the catalyst zone of the reactor, and thus the catalyst itself. The selected temperature thus exists in the catalyst zone of the reactor upon the occasion when the Formula (II) dinitrile compound and the ammonia component are contacted in the presence of the catalyst.

The reaction may be run at ambient pressure, or at a pressure of up to about 75 atm or up to about 150 atm (up to about 7.6 MPa or up to about 15.2 MPa), or at a pressure in the range of about 1 to about 10 atm (about 0.10 to about 1.0 MPa), or at a pressure in the range of about 1 to about 2 atm (about 0.10 to about 0.20 MPa). The reaction may be run for a length of time of a minute or less, or for a length of time of about 5 to about 10 seconds, or of about 1 to about 2 seconds, or of less than one second. In all cases, however, the reaction is carried out at a temperature and pressure and for a time that is sufficient to obtain liquid-phase production of a Formula (I) diaminopyridine reaction product.

In various embodiments, the amount of ammonia fed to the reactor may be about 1 molar equivalent or more, or about 10 molar equivalents or more, or about 25 molar equivalents or more, and yet about 700 molar equivalents or less, about 400 molar equivalents or less, or about 300 molar equivalents or less; or may be in the range of from about 1 molar equivalent to about 700 molar equivalents, or in the range of from about 10 molar equivalents to 400 molar equivalents, or in the range of from about 25 molar equivalents to 300 molar equivalents, per molar equivalent of Formula (II) dinitrile compound that is fed in. In yet other embodiments, a diaminopyridine compound may be produced at a concentration in the range of from about 1 to about 400 molar equivalents per molar equivalent of the Formula (II) dinitrile compound used in the reaction.

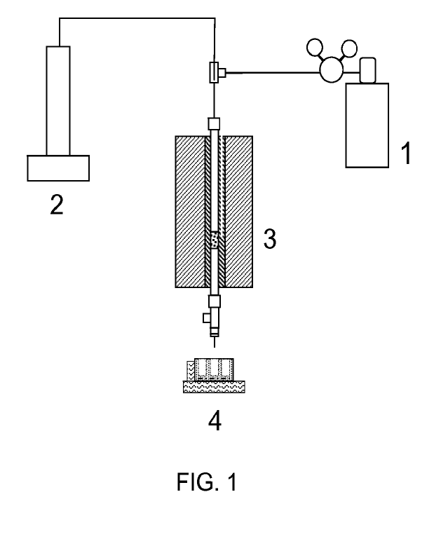

Reactors suitable for use in the processes hereof include fixed-bed reactors, and pipe, tubular or other plug-flow reactors and the like in which the catalyst particles are held in place and do not move with respect to a fixed residence frame; or fluidized bed reactors. Reactants may be flowed into and through reactors such as these on a continuous basis to give a corresponding continuous flow of product at the downstream end of the reactor. These and other suitable reactors are more particularly described, for example, in Fogler, Elements of Chemical Reaction Engineering, 2nd Edition, Prentice-Hall Inc. (1992). One example of a continuous, fixed-bed, liquid-phase reactor as used in an embodiment of the processes hereof is shown in FIG. 1. In a reactor such as shown in FIG. 1, in-flow lines for the ammonia component (1) and dinitrile feed (2) are heat traced to keep reactants at a suitable temperature, and the temperature of the catalyst zone (3) is controlled by a separate heating element at that location. The diaminopyridine product is collected from the reactor effluent (4).

A compound of Formula (I) (a “Pyridine Product”), after being produced for example in the manner as described above, may, as desired, be isolated and recovered. The Pyridine Product may also, however, be subjected with or without recovery from the reaction mixture to further steps to convert it to another product such as another compound (such as a type useful, for example, as a monomer), or an oligomer or a polymer. Another embodiment of a process hereof thus provides a process for converting a Pyridine Product, through a reaction (including a multi-step reaction), into another compound, or into an oligomer or a polymer. A Pyridine Product may be made by a process such as described above, and then converted, for example, by being subjected to a polymerization reaction to prepare an oligomer or polymer therefrom, such as those having amide functionality, imide functionality, or urea functionality, or a pyridobisimidazole-2,6-diyl(2,5-dihydroxy-p-phenylene) polymer.

A Pyridine Product such as a diaminopyridine may be converted into a polyamide oligomer or polymer by reaction with a diacid (or diacid halide) in a process in which, for example, the polymerization takes place in solution in an organic compound that is liquid under the conditions of the reaction, is a solvent for both the diacid(halide) and the diaminopyridine, and has a swelling or partial salvation action on the polymeric product. The reaction may be effected at moderate temperatures, e.g. under 100° C., and is preferably effected in the presence of an acid acceptor that is also soluble in the chosen solvent. Suitable solvents include methyl ethyl ketone, acetonitrile, N,N-dimethylacetamide dimethyl formamide containing 5% lithium chloride, and N-methyl pyrrolidone containing a quaternary ammonium chloride such as methyl tri-n-butyl ammonium chloride or methyl-tri-n-propyl ammonium chloride. Combination of the reactant components causes generation of considerable heat and the agitation, also, results in generation of heat energy. For that reason, the solvent system and other materials are cooled at all times during the process when cooling is necessary to maintain the desired temperature. Processes similar to the foregoing are described in U.S. Pat. No. 3,554,966; U.S. Pat. No. 4,737,571; and CA 2,355,316.

A Pyridine Product such as a diaminopyridine may also be converted into a polyamide oligomer or polymer by reaction with a diacid (or diacid halide) in a process in which, for example, a solution of the diaminopyridine in a solvent may be contacted in the presence of an acid acceptor with a solution of a diacid or diacid halide, such as a diacid chloride, in a second solvent that is immiscible with the first to effect polymerization at the interface of the two phases. The diaminopyridine may, for example, be dissolved or dispersed in a water containing base with the base being used in sufficient quantities to neutralize the acid generated during polymerization. Sodium hydroxide may be used as the acid acceptor. Preferred solvents for the diacid(halide) are tetrachloroethylene, methylenechloride, naphtha and chloroform. The solvent for the diacid(halide) should be a relative non-solvent for the amide reaction product, and be relatively immiscible in the amine solvent. A preferred threshold of immiscibility is as follows: an organic solvent should be soluble in the amine solvent not more than between 0.01 weight percent and 1.0 weight percent. The diaminopyridine, base and water are added together and vigorously stirred. High shearing action of the stirrer is important. The solution of acid chloride is added to the aqueous slurry. Contacting is generally carried out at from 0° C. to 60° C., for example, for from about 1 second to 10 minutes, and preferably from 5 seconds to 5 minutes at room temperature. Polymerization occurs rapidly. Processes similar to the foregoing are described in U.S. Pat. No. 3,554,966 and U.S. Pat. No. 5,693,227.

A Pyridine Product such as a diaminopyridine may also be converted into a polyimide oligomer or polymer by reaction with a tetraacid (or halide derivative thereof) or a dianhydride in a process in which each reagent (typically in equimolar amounts) is dissolved in a common solvent, and the mixture is heated to a temperature in the range of 100˜250° C. until the product has a viscosity in the range of 0.1˜2 dL/g. Suitable acids or anhydrides include benzhydrol 3,3′,4,4′-tetracarboxylic acid, 1,4-bis(2,3-dicarboxyphenoxy) benzene dianhydride, and 3,3′,4,4′-benzophenone tetracarboxylic acid dianhydride. Suitable solvents include cresol, xylenol, diethyleneglycol diether, gamma-butyrolactone and tetramethylenesulfone. Alternatively, a polyamide-acid product may be recovered from the reaction mixture and advanced to a polyimide by heating with a dehydrating agent such as a mixture of acetic anhydride and beta picoline. Processes similar to the foregoing are described in U.S. Pat. No. 4,153,783; U.S. Pat. No. 4,736,015; and U.S. Pat. No. 5,061,784.

A Pyridine Product such as a diaminopyridine may also be converted into a polyurea oligomer or polymer by reaction with a polyisocyanate, representative examples of which include toluene diisocyanate; methylene bis (phenyl isocyanates); hexamethylene diisocyanates; phenylene diisocyanates. The reaction may be run in solution, such as by dissolving both reagents in a mixture of tetramethylene sulfone and chloroform with vigorous stirring at ambient temperature. The product can be worked up by separation with water, or acetone and water, and then dried in a vacuum oven. Processes similar to the foregoing are described in U.S. Pat. No. 4,451,642 and Kumar, Macromolecules 17, 2463 (1984). The polyurea forming reaction may also be run under interfacial conditions, such as by dissolving the diaminopyridine in an aqueous liquid, usually with an acid acceptor or a buffer. The polyisocyanate is dissolved in an organic liquid such as benzene, toluene or cyclohexane. The polymer product forms at the interface of the two phases upon vigorous stirring. Processes similar to the foregoing are described in U.S. Pat. No. 4,110,412 and Millich and Carraher, Interfacial Syntheses, Vol. 2, Dekker, New York, 1977. A diaminopyridine may also be converted into a polyurea by reaction with phosgene, such as in an interfacial process as described in U.S. Pat. No. 2,816,879.

A Pyridine Product such as a tetraaminopyridine may be converted to a pyridobisimidazole-2,6-diyl(2,5-dihydroxy-p-phenylene) polymer by polymerizing a 2,5-dihydroxyterephthalic acid with the trihydrochloride-monohydrate of tetraaminopyridine in strong polyphosphoric acid under slow heating above 100° C. up to about 180° C. under reduced pressure, followed by precipitation in water, as disclosed in U.S. Pat. No. 5,674,969 (which is incorporated in its entirety as a part hereof for all purposes); or by mixing the monomers at a temperature from about 50° C. to about 110° C., and then 145° C. to form an oligomer, and then reacting the oligomer at a temperature of about 160° C. to about 250° C. as disclosed in U.S. Patent Publication 2006/0287475 (which is incorporated in its entirety as a part hereof for all purposes). The pyridobisimidazole-2,6-diyl(2,5-dihydroxy-p-phenylene) polymer so produced may be, for example, a poly(1,4-(2,5-dihydroxy) phenylene-2,6-pyrido[2, 3-d: 5,6-d′]bisimidazole) polymer, or a poly[(1,4-dihydrodiimidazo[4,5-b:4′,5′-e]pyridine-2,6-diyl) (2,5-dihydroxy-1,4-phenylene)] polymer. The pyridobisimidazole portion thereof may, however, be replaced by any one or more of a benzobisimidazole, benzobisthiazole, benzobisoxazole, pyridobisthiazole and a pyridobisoxazole; and the 2,5-dihydroxy-p-phenylene portion thereof may be replaced by the derivative of one or more of isophthalic acid, terephthalic acid, 2,5-pyridine dicarboxylic acid, 2,6-naphthalene dicarboxylic acid, 4,4′-diphenyl dicarboxylic acid, 2,6-quinoline dicarboxylic acid, and 2,6-bis(4-carboxyphenyl)pyridobisimidazole.

### EXAMPLES

The advantageous attributes and effects of the processes hereof may be seen in a series of examples (Examples 1 and 2), as described below. The embodiments of these processes on which the examples are based are representative only, and the selection of those embodiments to illustrate the invention does not indicate that conditions, regimes, steps, techniques, configurations, protocols, materials or reactants not described in these examples are not suitable for practicing these processes, or that subject matter not described in these examples is excluded from the scope of the appended claims and equivalents thereof.

Materials.

The following materials were used in the examples. Commercial reagents, such as glutaronitrile (99%), ethanol (99.5%), and 2,6-diaminopyridine (98%), were obtained from Aldrich Chemical Company (Milwaukee, Wis., USA), and used as received unless otherwise noted. Palladium (0.5 weight percent on alumina as 1/16-inch round beads) catalyst was obtained from Engelhard Corporation (now BASF Catalysts LLC, Florham Park, N.J., USA), and used as received unless otherwise noted. Anhydrous ammonia (99.99%) was obtained from MG Industries (Malvern, Pa., USA) and used as received.

Methods

In these examples, the following protocol was used (except as noted in the description of a particular example): the reactions were carried out in a custom fixed-bed liquid phase reactor fabricated from ⅜-inch (0.95-cm) 316 S.S. tubing (numerical references below being to FIG. 1). The reactor was operated under continuous flow of mixtures of anhydrous ammonia (1) and organic reactants. The organic reactants were optionally dissolved in a solvent, such as ethanol, and were metered as a liquid by a syringe pump (Isco Model 100 DM) (2) and heated to reaction temperature by passing the liquid feed through a heated injector and combining it with the heated ammonia gas. The ammonia was metered with a mass flow controller (Brooks Model 5850E). The inlet lines and liquid injector were heat traced with electrical heating tape to pre-heat the reactor feeds prior to contacting the catalyst reaction zone (3). The reactor and catalyst reaction zone were heated with an electrical tube furnace. The reactor effluent was passed through a chiller and then a syringe needle into vented and chilled sample vials where the liquid products were collected (4), and unreacted ammonia was vented to a fume hood containing the entire apparatus. A circulating bath was used to chill these sample recovery vials.

The meaning of abbreviations as used in the examples is as follows: “bp” means boiling point, “cm” means centimeter(s), “DAP” means 2,6-diaminopyridine, “g” means gram(s), “GN” means glutaronitrile, “LDL” means lower-detection limit, “min” means minute(s), “mL” means milliliter(s), “MHz” means megahertz, “NMR” means nuclear magnetic resonance spectroscopy, “mol” means mole, “mmol” means millimole(s), “μmol” means micromole(s), “Pd/Al₂O₃” means palladium on alumina catalyst, “scc” means standard cubic centimeter (cubic centimeters at standard conditions of temperature and pressure), “temp” means temperature, and “TLC” means thin-layer chromatography.

In Examples 1-2, qualitative evidence for DAP formation was determined by TLC (silica gel 60 F₂₅₄ plates, 2.5 cm×7.5 cm) and/or ¹H NMR spectral analysis, with comparison of crude product mixtures with authentic material as specified. For TLC, the LDL was confirmed to be less than 1 μmol/mL. Percent conversion was estimated based on ¹H NMR spectral integration, recorded at 500 MHz unless otherwise specified, of 2,6-diaminopyridine (DAP) produced in the reaction. The temperature reported is the temperature at the catalyst zone of the reactor.

### Examples 1-2

These examples demonstrate qualitative fixed-bed liquid-phase conversion of GN to DAP.

The reactor zone was charged with 2 g catalyst and pre-heated to approximately 160° C. The reactor inlet lines were pre-heated to approximately 160° C. Once the temperatures had equilibrated, anhydrous ammonia flow was set at 1000 scc ammonia per min. A solution of glutaronitrile (bp 285-287° C.) (25.0 g, 265.62 mmol) in ethanol (75.0 g, 1.63 mol) was loaded into the syringe pump and fed to the reaction zone at flow rates and reactor temperatures per those indicated in Table 1. Following reaction at the specified condition, DAP was detected by TLC and/or ¹H NMR analysis in each example.

### Table 1.

[symbol]

Where a range of numerical values is recited herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value.

In this specification, unless explicitly stated otherwise or indicated to the contrary by the context of usage, where an embodiment of the subject matter hereof is stated or described as comprising, including, containing, having, being composed of or being constituted by or of certain features or elements, one or more features or elements in addition to those explicitly stated or described may be present in the embodiment. An alternative embodiment of the subject matter hereof, however, may be stated or described as consisting essentially of certain features or elements, in which embodiment features or elements that would materially alter the principle of operation or the distinguishing characteristics of the embodiment are not present therein. A further alternative embodiment of the subject matter hereof may be stated or described as consisting of certain features or elements, in which embodiment, or in insubstantial variations thereof, only the features or elements specifically stated or described are present.

In this specification, unless explicitly stated otherwise or indicated to the contrary by the context of usage,
(a) amounts, sizes, ranges, formulations, parameters, and other quantities and characteristics recited herein, particularly when modified by the term “about”, may but need not be exact, and may also be approximate and/or larger or smaller (as desired) than stated, reflecting tolerances, conversion factors, rounding off, measurement error and the like, as well as the inclusion within a stated value of those values outside it that have, within the context of this invention, functional and/or operable equivalence to the stated value;
(b) all numerical quantities of parts, percentage or ratio are given as parts, percentage or ratio by weight;
(c) use of the indefinite article “a” or “an” with respect to a statement or description of the presence of an element or feature of this invention, does not limit the presence of the element or feature to one in number; and
(d) the words “include”, “includes” and “including” are to be read and interpreted as if they were followed by the phrase “without limitation” if in fact that is not the case.

What is claimed is:

1. A process for the synthesis of a compound as represented by the structure of the following Formula (I): 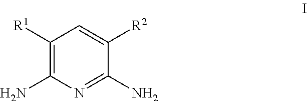
comprising (a) providing a compound as represented by the structure of the following Formula (II): 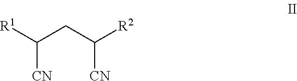
in the form of a liquid; (b) providing an ammonia component selected from the group consisting of: neat liquid ammonia, a mixture of liquid ammonia and a solvent, and ammonia gas; (c) heating a heterogeneous dehydrogenation catalyst; and (d) contacting the Formula (II) compound and the ammonia component in the presence of the catalyst to produce a Formula (I) product; wherein R¹ and R² are each independently selected from H and a hydrocarbyl group; and wherein the process is a continuous process.

2. A process according to claim 1 wherein a hydrocarbyl group is selected from the groups consisting of
a C₁˜C₁₂ straight-chain or branched, saturated or unsaturated, substituted or unsubstituted, aliphatic hydrocarbyl group; and
a C₃˜C₁₂ cyclic, saturated or unsaturated, substituted or unsubstituted, aliphatic hydrocarbyl group.

3. A process according to claim 1 wherein one or both of R¹ and R² are selected from a C₁˜C₄ straight-chain or branched, saturated or unsaturated, substituted or unsubstituted, aliphatic hydrocarbyl group; and H.

4. A process according to claim 1 wherein R¹ and R² are both H.

5. A process according to claim 1 wherein the catalyst is heated to a temperature in the range of from about 125° C. to about 300° C.

6. A process according to claim 1 wherein the catalyst is heated to a temperature in the range of from about 150° C. to about 175° C.

7. A process according to claim 1 which is run at a pressure of up to about 15.2 MPa.

8. A process according to claim 1 wherein the heterogeneous dehydrogenation catalyst comprises at least one metal or metal salt and a support, wherein the metal, or the metal of a salt, is selected from elements of Groups IVA, VA, VIA, VIIA, VIII, IB and/or IIB of the Periodic Table.

9. A process according to claim 8 wherein the metal, or the metal of a salt, is selected from one or more members of the group consisting of iron, cobalt, nickel, ruthenium, rhodium, palladium, osmium, iridium, platinum, copper. and rhenium.

10. A process according to claim 8 wherein the metal is a sponge metal catalyst.

11. A process according to claim 8 wherein the metal, or the metal of a salt, is selected from one or more members of the group consisting of palladium and platinum; and the support comprises one or more materials selected from the group consisting of alumina, silica and activated carbon.

12. A process according to claim 8 wherein the support comprises one or more materials selected from the group consisting of alumina, titania, cobaltic oxide, zirconia, ceria, molybdenum oxide, tungsten oxide, silica, silicalite, a zeolite or zeotype material, activated carbon, coke and charcoal.

13. A process according to claim 8 wherein R¹ and R² are both H; and the heterogeneous dehydrogenation catalyst comprises palladium or platinum, and/or a support comprising one or more materials selected from the group consisting of alumina, silica and activated carbon.

14. A process according to claim 1 wherein the Formula (II) compound is dissolved in a solvent.

15. A process according to claim 1 which is run for a time of less than one minute.

16. A process according to claim 1 wherein the amount of ammonia fed to the reactor is in the range of from about 1 molar equivalent to about 700 molar equivalents per molar equivalent of Formula (II) dinitrile compound that is fed in.

17. A process according to claim 1 further comprising a step of subjecting the Formula (I) compound to a reaction to prepare therefrom a compound, oligomer or polymer.

18. A process according to claim 17 wherein a polymer prepared comprises amide functionality, imide functionality, or urea functionality.

19. A process according to claim 17 wherein a polymer prepared comprises a pyridobisimidazole-2,6-diyl(2,5-dihydroxy-p-phenylene) polymer, or a poly[(1,4-dihydrodiimidazo[4,5-b:4′,5′-e]pyridine-2,6-diyl) (2,5-dihydroxy-1,4-phenylene)] polymer.
